(19) United States
(12) Patent Application Publication
(10) Pub. No.: US 2015/0044144 A1
(43) Pub. Date: Feb. 12, 2015

(54) DISINFECTANT SOLUTION

(71) Applicant: RAYMAT MATERIALS, INC., Pleasanton, CA (US)

(72) Inventors: Jibing Lin, Pleasanton, CA (US); Ning Liu, Fremont, CA (US)

(73) Assignee: RAYMAT MATERIALS, INC., Pleasanton, CA (US)

(21) Appl. No.: 14/376,434

(22) PCT Filed: Mar. 6, 2013
(86) PCT No.: PCT/US13/29225

§ 371 Date: Aug. 4, 2014

Related U.S. Application Data
(60) Provisional application No. 61/607,556, filed on Mar. 6, 2012.

(51) Int. Cl. A01N 25/00; A61K 33/20; A61K 47/02; A01N 59/00

(52) U.S. Cl. CPC A01N 25/00; A01N 59/00; A61K 33/20; A61K 47/02
USPC 424/10.3

(57) ABSTRACT

The present invention relates to a disinfectant solution, comprising an oxidative and reductive potential (ORP) solution and a colorant. A disinfectant article, a method of forming the disinfectant solution and use of the disinfectant solution are also provided.

### CROSS-REFERENCE TO RELATED APPLICATIONS

The present application claims the benefit of U.S. Provisional Patent Application No. 61/607,556, filed on Mar. 6, 2012, which is incorporated herein by reference in its entirety.

### FIELD OF THE INVENTION

The present invention relates to a disinfectant solution. The present invention further relates to a disinfectant solution comprising a colorant.

### BACKGROUND

Oxidative reductive potential (ORP) solution, also known as super-oxidized water or redox potential water, has been used as eco-friendly, non-toxic disinfectant. However, when visually observable colorants are needed to indicate the presence of disinfectant in the super-oxidized water, only very few colorant choices are available as most of synthetic dyes and natural colorants can not survive in the super-oxidized water. The choices are further limited if food and drug applications are involved to meet FDA standards. A person skilled in the art recognizes that no colorant can be used in ORP solution. Therefore, there are needs to identify colorants suitable for marking and develop disinfectant solution comprising colorants.

### SUMMARY OF THE INVENTION

One aspect of the present disclosure is directed to a disinfectant solution, comprising an oxidative and reductive potential (ORP) solution and a colorant.

In certain embodiments, the colorant is dissolved in the ORP solution.

In certain embodiments, the colorant renders the disinfectant solution a visible color.

In certain embodiments, the colorant is potassium permanganate.

In certain embodiments, the concentration of potassium permanganate is between a minimal value and a maximal value, wherein the minimal value is about 1 ppm and the maximal value is about 10%. In certain embodiments, the minimal value is selected from the group consisting of 1 ppm, 10 ppm, 20 ppm, 30 ppm, 40 ppm, 50 ppm, 60 ppm, 70 ppm, 80 ppm, 90 ppm, and 100 ppm. In certain embodiments, the maximal value is selected from the group consisting of 10%, 9%, 8%, 7%, 6%, 5%, 4%, 3%, 2%, 1%, and 2000 ppm. In certain embodiments, concentration of potassium permanganate is about 1-2000 ppm, about 10-2000 ppm, about 20-2000 ppm, about 30-2000 ppm, about 40-2000 ppm, about 50-2000 ppm, about 60-2000 ppm, about 70-2000 ppm, about 80-2000 ppm, about 90-2000 ppm, or about 100-2000 ppm.

In certain embodiments, pH value of the disinfectant solution is between about 3 and about 9. In certain embodiments, pH value of the disinfectant solution is between about 6 and about 8. In certain embodiments, pH value of the disinfectant solution is between about 6.5 and about 7.5.

In certain embodiments, the potential of disinfectant solution is between +500 mV and +1250 mV. In certain embodiments, the potential of disinfectant solution is between +600 mV and +1100 mV. In certain embodiments, the potential of disinfectant solution is between +700 mV and +800 mV.

In certain embodiments, the ORP solution contains at least one species having disinfectant ability. In certain embodiments, the species is chlorine, bromine or ozone. In certain embodiments, chlorine is free available chlorine. In certain embodiments, the concentration of the free available chlorine is about 5 ppm to about 6%. In certain embodiments, the concentration of the free available chlorine is about 20 ppm to about 1000 ppm.

In certain embodiments, the disinfectant solution further comprises an additive or a bleaching agent. In certain embodiments, the additive is selected from the group consisting of surfactant, detergent, cleaning agent, perfume, and scent-producing agent. In certain embodiments, the bleaching agent is chlorine-containing bleaching agent. In certain embodiments, the chlorine-containing bleaching agent is selected from the group consisting of chlorine, hypochlorites, N-chloro compounds, chlorine dioxide, sodium hypochlorite, hypochlorous acid, calcium hypochlorite, bleach liquor (aqueous solution of calcium hypochlorite and calcium chloride), bleaching powder (mixture of calcium hypochlorite, calcium hydroxide, calcium chloride, and hydrates thereof), dibasic magnesium hypochlorite, lithium hypochlorite, chlorinated trisodium phosphate and any combination thereof.

In certain embodiments, the pH of the bleaching agent is from about 8 to about 10, and the potential of the solution is from about +700 mV to about +800 mV.

In certain embodiments, the disinfectant solution is stable up to about 90 days, about 180 days, about 1 year, or about 2 years.

In certain embodiments, the solution can be in the state of liquid, solid, aerosol, slurry or gel.

In certain embodiments, the solution is packaged or sealed in a container.

In certain embodiments, the OPR is in a first container and the colorant is in a second container.

Another aspect of the present disclosure is directed to a disinfectant article, comprising a substrate and the above disinfectant solution. In certain embodiments, the substrate can be nonwoven materials, woven materials, compound materials, or knit materials. In certain embodiments, the solution is dispensed, impregnated, coated, covered or applied to the substrate. In certain embodiments, the solution is packed in a first container and the substrate is placed in a second container. In certain embodiments, the article is packed in the dispenser.

Another aspect of the present disclosure is directed to a method of forming the above disinfectant solution, comprising the steps of 1) providing oxidative and reductive potential (ORP) solution, and 2) dissolving a colorant into ORP solution.

Another aspect of the present disclosure is directed to use of the above disinfectant solution, thereof in killing microorganisms, disinfecting surfaces and milking equipment; working as anti-infective agent; blocking inflammatory process, speeding the healing of burns, wounds, and diabetic ulcers; or treating periodontal diseases and skin disorders.

Another aspect of the present disclosure is directed to use of the above disinfectant solution, thereof in medical device sterilization, food sterilization, hospitals, consumer households, agriculture, or anti-bioterrorism.

### DESCRIPTION OF THE DRAWINGS

FIG. 1. UV-vis spectra of disinfectant solution comprising 300 ppm potassium permanganate and ORP solution containing high and low concentration of free active chlorine after different time period

FIG. 2. UV-vis spectra of disinfectant solution comprising 300 ppm FeCl₃ and ORP solution containing high concentration of free active chlorine, and control solution

FIG. 3. UV-vis spectra of disinfectant solution comprising 300 ppm Copper (II) Sulfate pentahydrate and ORP solution containing high concentration of free active chlorine, and control solution

FIG. 4. UV-vis spectra of disinfectant solution comprising 300 ppm ammonium ferric (iron III) citrate and ORP solution containing high concentration of free active chlorine, and control solution

FIG. 5. UV-vis spectra of disinfectant solution comprising 300 ppm Eosine Free Acid Solvent Red 43 D&C red 21 and ORP solution containing low concentration of free active chlorine, and control solution

FIG. 6. UV-vis spectra of disinfectant solution comprising 300 ppm Eosine YS Acid Red 87 D&C red 22 and ORP solution containing high concentration of free active chlorine, and control solution

FIG. 7. UV-vis spectra of disinfectant solution comprising 300 ppm Food dye FD&C red 40 and ORP solution containing high and low concentration of free active chlorine after different time period, and control solution

FIG. 8. The relationship between ORP value and killing time of E. Coli bacteria

### DETAILED DESCRIPTION OF THE INVENTION

One aspect of the present disclosure is related to a disinfectant solution, comprising an oxidative and reductive potential (ORP) solution and a colorant.

### Oxidative Reductive Potential (ORP)

Oxidative reductive potential (ORP) solution or water, also known as super-oxidized water or redox potential water, can be used as a non-toxic disinfectant to eradicate microorganisms, including bacteria, viruses and spores, in variety of settings. For example, ORP water may be applied in the dairy farm to disinfect surface and milking equipment. Advantageously, ORP water is environmentally safe and, thus, avoids the need for costly disposal procedures. ORP water also has application in wound care, medical device sterilization, food sterilization, hospitals, consumer households and anti-bioterrorism.

ORP solution (or interchangeably ORP water) referred herein is a liquid or solution containing ORP and reflects antimicrobial potential of the solution or water. ORP solution can be obtained by electrolysis of saline solution through an electrolytic cell, comprising an anode chamber, a cathode chamber and a separator. When currents pass through the saline solution, the anode oxidizes chloride ions (Cl⁻), and Cl₂ gas is produced. However, at the cathode, instead of sodium ions being reduced to sodium metal, water molecules are reduced to hydroxide ions (OH⁻) and hydrogen gas (H₂). The overall result of the electrolysis is the production of chlorine gas and aqueous sodium hydroxide (NaOH) solution. The separator prevents the Cl₂ produced at the anode in this cell from coming into contact with the NaOH that accumulates at the cathode. When the seperator is removed from the cell, the products of the electrolysis of aqueous sodium chloride react to form ORP solution containing sodium hypochlorite.

Oxidation-reduction potential used herein is the potential (voltage) at which oxidation occurs at the anode (positive) and reduction occurs at the cathode (negative) of an electrochemical cell. Without been bounded to any theory, from a microbial perspective, an oxidizing chemical pulls electrons away from the microbial cell membrane, causing it to become destabilized and leaky. Disruption of the integrity of the cell membrane leads to rapid death of the microorganism.

The potential of ORP solution can be measured by standard techniques (e.g., ORP sensors) including, for example, by measuring the electrical potential in millivolts of the ORP solution relative to standard reference silver/silver chloride electrode. This potential is a measure of the tendency (i.e., the potential) of a solution to either accept or transfer electrons that is sensed by a metal electrode and compared with a reference electrode in the same solution.

ORP solution is known to be a reliable indicator of bacteriological water quality. The killing time of E. Coli bacteria is a function of ORP value. With a value of 600 mV, the life of the bacteria is almost 2 minutes; at 650 mV it reduces to 30 seconds. Above 700 mV the bacteria is killed within a few seconds (see FIG. 8). ORP value of the solution has been proven to be more meaningful than measuring the concentration of residue or total species in the solution.

The measurement of ORP by ORP sensors can be coupled with pH sensors in commercially available systems to monitor and track disinfectant levels of ORP solution. Certain systems further provide automated need-based injection of hypochlorite (or other oxidizing disinfectants) and acid (e.g., citric acid (e.g., food-grade), muriatic acid, or phosphoric acid).

ORP solution may contain chlorine (e.g., free chlorine and bound chlorine), bromine and ozone. The presence of one or more of these species contributes to the disinfectant ability of the ORP water solution to kill a variety of microorganisms, such as bacteria and fungi, as well as viruses. Free chlorine typically includes, but is not limited to, hypochlorous acid (HClO), hypochlorite ions (ClO⁻), sodium hypochlorite (NaOCl), chloride ion (Cl⁻), chlorite ions (ClO₂⁻), chlorine dioxide (ClO₂), dissolved chlorine gas (Cl₂), and other radical chlorine species. The ratio of hypochlorous acid to hypochlorite ion is dependent upon pH. In certain embodiments, hypochlorite ions and hypochlorous acid are both effective for disinfection. In preferred embodiments, hypochlorous acid is effective for disinfection all of the total free chlorine would be in the form of hypochlorous acid if the pH was low enough. At a pH of 7.4, hypochlorous acid levels are from about 25 ppm to about 75 ppm. Temperature also impacts the ratio of the free chlorine component.

### Colorant

The term “colorant” used herein in refers to a visually observable solute or substance dissolvable in a solution, which, once dissolved, distinguishes the solution from others or imparts a color to the solution and therefore change the color of the solution (e.g., turning from colorless to a visible color). The colorant used herein further meets the following requirements. Under certain circumstances, visually observable colorants are required in the ORP disinfectant solution to mark the presence of ORP solution. The colorants of the present disclosure must be water soluble or ORP water soluble and impart a visible color to the solution once dissolved in contrast to colorlessness. The colorants must be sustainable and stable in the ORP environment and not be subject to oxidation. As ORP water may be used for medical applications or food disinfection, the colorants should, optionally, be in food or drug applicable categories approved by FDA.

It is unexpectedly discovered that potassium permanganate meets the requirements as a colorant for ORP solutions. As a strong oxidizing agent, potassium permanganate is water soluble, stable in the ORP environment, and renders intense purple color and therefore acts as a good candidate as stable colorant for ORP water. Potassium permanganate itself is a disinfectant being antiseptic and antimicrobial. Potassium permanganate has been used to disinfect drinking water and treat gonorrhea. Its current application is in the control of nuisance organisms such as Zebra mussels in fresh water collection and treatment systems. It also finds applications in treating ulcers, mild pompholyx, dermatitis, fungal infections of the hands or feet, removing iron and hydrogen sulfide from well water via a “Manganese Greensand” Filter, inactivating the poison strychnine, for fruit preservation and in survival kit. Thus, potassium permanganate can be used in food or drug related applications.

In addition to qualifying as a colorant for ORP solutions disclosed herein, the presence of potassium permanganate in the ORP solution further brings these effects: 1) improving or enhancing the overall disinfection function of the solution; 2) stabilizing the solution and extending the shell life; and 3) adding observable vivid color (purple) into the ORP solution.

In certain embodiments, the concentration of potassium permanganate is between a minimal value and a maximal value, wherein the minimal value is about 1 ppm and the maximal value is about 10%. In certain embodiments, the minimal value is selected from the group consisting of 1 ppm, 10 ppm, 20 ppm, 30 ppm, 40 ppm, 50 ppm, 60 ppm, 70 ppm, 80 ppm, 90 ppm, and 100 ppm. In certain embodiments, the maximal value is selected from the group consisting of 10%, 9%, 8%, 7%, 6%, 5%, 4%, 3%, 2%, 1%, and 2000 ppm. In certain embodiments, concentration of potassium permanganate is about 1-2000 ppm, about 10-2000 ppm, about 20-2000 ppm, about 30-2000 ppm, about 40-2000 ppm, about 50-2000 ppm, about 60-2000 ppm, about 70-2000 ppm, about 80-2000 ppm, about 90-2000 ppm, or about 100-2000 ppm.

### Additives & Bleaching Agents

The disinfectant solution administered in accordance with the present disclosure optionally can contain additives suitable, e.g., for the household and workplace cleaning environment. Suitable additives can include, e.g., surfactants, such as detergents and cleaning agents. Perfumes or other scent-producing compounds also can be included to enhance consumer reception of the disinfectant solution.

For some applications, the disinfectant solution optionally can contain a bleaching agent. The bleaching agent can include, e.g., a compound that lightens or whitens a substrate. The disinfectant solution containing a bleaching agent can be used in home laundering to disinfect and sterilize bacteria and germs as well as brighten clothing. Suitable bleaching agents include, but are not limited to, chlorine-containing bleaching agents. Mixtures of bleaching agents also can be added to the disinfectant solution. The bleaching agent can be added in the form of an aqueous solution to the disinfectant solution.

Suitable chlorine-containing bleaching agents can include, e.g., chlorine, hypochlorites, N-chloro compounds, and chlorine dioxide. Preferably, the chlorine-containing bleaching agent added to the disinfectant solution is sodium hypochlorite or hypochlorous acid. Other suitable chlorine-containing bleaching agents include, e.g., chlorine, calcium hypochlorite, bleach liquor (e.g., aqueous solution of calcium hypochlorite and calcium chloride), bleaching powder (e.g., mixture of calcium hypochlorite, calcium hydroxide, calcium chloride, and hydrates thereof), dibasic magnesium hypochlorite, lithium hypochlorite, chlorinated trisodium phosphate and mixtures thereof.

The addition of a bleaching agent to the disinfectant solution can be carried out in any suitable manner. For instance, an aqueous solution containing a bleaching agent can be prepared using household bleach (e.g., Clorox® bleach) or other suitable source of chlorine-containing bleaching agent or other bleaching agent. The bleaching agent solution can then be combined with the disinfectant solution.

The bleaching agent can be added to the disinfectant solution in any suitable amount. Preferably, the disinfectant solution containing a bleaching agent is non-irritating to human or animal skin. The total chloride ion content of the disinfectant solution containing a chlorine-containing bleaching agent can be from about 1000 ppm to about 5000 ppm, e.g., from about 1000 ppm to about 3000 ppm. The pH of the disinfectant solution containing a chlorine-containing bleaching agent is preferably from about 8 to about 10, and the oxidative-reductive potential of the solution is preferably from about +700 mV to about +800 mV.

### Disinfectant Article

The disinfectant solution also can be applied on a disinfectant or a cleaning article comprising a substrate and the disinfectant solution described herein, wherein the disinfectant solution is dispensed onto the substrate. The disinfectant solution can be impregnated, coated, covered or otherwise applied to the disinfectant article substrate. Preferably, the substrate is pretreated with the disinfectant solution before distribution of the disinfectant articles to end users. In one embodiment, the substrate is water insoluble.

In certain embodiments, the material for the substrate can be any suitable water-insoluble absorbent or adsorbent material. It should have sufficient wet strength, abrasivity, loft and porosity. Further, the substrate should not adversely impact the stability of the disinfectant solution. Examples include nonwoven substrates, woven substrates, compound substrates, knit substrate.

The substrate can have one or more layers. Each layer can have the same or different textures and abrasiveness. Differing textures can result from the use of different combinations of materials or from the use of different manufacturing processes or a combination thereof. The substrate should not dissolve or break apart in water. The substrate can thereby provide a vehicle for delivering the disinfectant solution to the surface to be treated.

The disinfectant solution can be dispensed, impregnated, coated, covered or otherwise applied to the substrate by any suitable method. For example, individual portions of substrate can be treated with a discrete amount of the disinfectant solution. Preferably, a mass treatment of a continuous web of substrate material with the disinfectant solution is carried out. The entire web of substrate material can be soaked in the disinfectant solution. Alternatively, as the substrate web is spooled, or even during creation of a nonwoven substrate, the disinfectant solution can be sprayed or metered onto the web. A stack of individually cut and sized portions of substrate can be impregnated or coated with the ORP water solution in its container by the manufacturer.

The disinfectant articles optionally can contain additional components to improve the properties of the disinfectant articles. For example, the disinfectant articles can further comprise hydrophilic polymers, hydrophobic polymers, surfactants, solvents, chelating agents, buffers, thickeners, fragrances, and mixtures thereof to improve the properties of the disinfectant articles. These optional components should not adversely impact the stability of the disinfectant solution.

The disinfectant articles of the present disclosure can be individually sealed with a heat-sealable or glueable thermoplastic overwrap (such as polyethylene, Mylar, and the like). The disinfectant articles can also be packaged as numerous, individual sheets for more economical dispensing. The disinfectant articles can be prepared by first placing multiple sheets of the substrate in a dispenser and then contacting the substrate sheets with the disinfectant solution of the present disclosure. Alternatively, the disinfectant articles can be formed as a continuous web by applying the disinfectant solution to the substrate during the manufacturing process and then loading the wetted substrate into a dispenser.

The dispenser includes, but is not limited to, a canister with a closure, or a tub with closure. The closure on the dispenser is to seal the disinfectant articles from the external environment and to prevent premature volatilization of the liquid ingredients.

The dispenser can be made of any suitable material that is compatible with both the substrate and the disinfectant solution. For example, the dispenser can be made of plastic, such as high density polyethylene, polypropylene, polycarbonate, polyethylene terephthalate (PET), polyvinyl chloride (PVC), or other rigid plastics.

The continuous web of the disinfectant articles can be threaded through a thin opening in the top of the dispenser, most preferably, through the closure. A means of sizing the desired length or size of the disinfectant articles from the web can then be desirable. A knife blade, serrated edge, or other means of cutting the web to desired size can be provided on the top of the dispenser, for non-limiting example, with the thin opening actually doubling in duty as a cutting edge. Alternatively, the continuous web of the disinfectant articles can be scored, folded, segmented, perforated or partially cut into uniform or non-uniform sizes or lengths, which would then obviate the need for a sharp cutting edge. Further, the disinfectant articles can be interleaved, so that the removal of one disinfectant article advances the next.

### Method of Forming the Disinfectant Solution

ORP solution can be prepared by reacting dilute sodium hydroxide solution with liquid or gaseous chlorine, accompanied by cooling. The basic principle of operation is to put water into a tank and add 50% sodium hydroxide until the strength of the caustic is approximately 6.75% (typical). Within a few batches, the amount of water and amount of sodium hydroxide to be added will be established and then lines may be drawn on the tank to show the operators how much water, and then sodium hydroxide, to add.

The 50% sodium hydroxide can be pumped into the tank or pulled into the system from the shipping container or a storage tank by partially closing the recycle tank outlet valve and opening the 50% caustic tank storage valve. After the sodium hydroxide is mixed, chlorine is added to the solution to react with the sodium hydroxide, except for a small amount of excess (0.2% by weight typical). Sodium hypochlorite strength can be varied by changing the strength of the diluted caustic.

ORP solution can also be obtained by electrolysis of saline solution such as sodium chloride through an electrochemical cell.

When certain percentage of ORP solution is made, potassium permanganate in the form of powder or concentrated solution is added into the ORP solution. Meantime, phosphate salts are also added to adjust the pH value of the solution.

In certain embodiments, the ORP solution is stored in a container and potassium permanganate in the form of powder or solution is stored in a separate container. Prior to the use or forming the disinfectant solution, the ORP solution and potassium permanganate are then mixed.

### Forms and Applications of Disinfectant Solution

The disinfectant solution may be used to disinfect and sterilize in any suitable manner. For example, to disinfect and sterilize medical or dental equipment, the equipment can be maintained in contact with the disinfectant solution for a sufficient period of time to reduce the level of organisms present on the equipment to a desired level.

For disinfection and sterilization of hard surfaces, the disinfectant solution can be applied to the hard surface directly from a container in which the disinfectant solution is stored. For example, the disinfectant solution can be poured, sprayed or otherwise directly applied to the hard surface. The disinfectant solution can then be distributed over the hard surface using a suitable substrate such as, for example, cloth, fabric or paper towel. In hospital applications, the substrate is preferably sterile. Alternatively, the disinfectant solution can first be applied to a substrate such as cloth, fabric or paper towel. The wetted substrate can then be contacted with the hard surface. Alternatively, the disinfectant solution can be applied to hard surfaces by dispersing the solution into the air as described herein. The disinfectant solution can be applied in a similar manner to humans and animals.

The disinfectant solution of the present disclosure alternatively can be dispersed into the environment through a gaseous medium, such as air. The disinfectant solution can be dispersed into the air by any suitable means. For example, the disinfectant solution can be formed into droplets of any suitable size and dispersed into a room.

For small scale applications, the disinfectant solution can be dispensed through a spray bottle that includes a standpipe and pump. Alternatively, the disinfectant solution can be packaged in aerosol containers. Aerosol containers can include the product to be dispensed, propellant, container, and valve. The valve can include both an actuator and dip tube. The contents of the container can be dispensed by pressing down on the actuator. The various components of the aerosol container should be compatible with the disinfectant solution. Suitable propellants can include a liquefied halocarbon, hydrocarbon, or halocarbon-hydrocarbon blend, or a compressed gas such as carbon dioxide, nitrogen, or nitrous oxide. Aerosol systems preferably yield droplets that range in size from about 0.15 μm to about 5 μm. In certain embodiment, the disinfectant solution can be dispersed into nanoparticle carriers or in the size of nanoparticles (e.g. 10-100 nm).

In addition to the state of the disinfectant solution in the form of liquid or aerosol, the disinfectant solution can also be in the form of solid (e.g., ice) or slurry or gel. For example, slurried ice can be produced from a disinfectant solution containing, for example, hyperochlorous acid slurried ice having rounded ice crystals within liquid. The slurry is formed by chilling the hypochlorous acid output solution to temperatures of approximately from −5 to 2° C.

### Stability

The disinfectant solution containing a colorant of the present disclosure can be stable for up to about 2 years (e.g., up to about 1 year, up to about 180 days, up to about 90 days).

Stability can be measured based on the ability of the disinfectant solution to remain suitable for one or more uses, for example, inhibiting mast cell degranulation, inhibiting cytokine secretion, decontamination, disinfection, sterilization, anti-microbial cleansing, and wound cleansing, for a specified period of time after its preparation under normal storage conditions (e.g., room temperature). The stability of the disinfectant solution administered in accordance with the present disclosure also can be measured by storage under accelerated conditions, e.g., from about 25° C. to about 60° C., wherein the disinfectant solution preferably is stable for a period of time disclosed herein.

Stability also can be measured based on the concentration over time of one or more species (or precursors thereof) present in solution during the shelf-life of the disinfectant solution. Preferably, the concentrations of one or more species, e.g., free chlorine, hypochlorous acid and one or more superoxidized water species are maintained at about 70% or greater of their initial concentration for at least about two months after preparation of the disinfectant solution. More preferably, the concentration of one or more of these species is maintained at about 80% or greater of their initial concentration for at least about two months after preparation of the disinfectant solution. Still more preferably, the concentration of one or more of such species is maintained at about 90% or greater, and most preferably is maintained at about 95% or greater, of their initial concentration for at least about two months after preparation of the disinfectant solution.

Stability also can be determined based on the reduction in the amount of organisms present in a sample following exposure to the disinfectant solution. Measuring the reduction of organism concentration can be made on the basis of any suitable organism including, e.g., bacteria, fungi, yeasts, or viruses. Suitable organisms can include, e.g., Escherichia coli, Staphylococcus aureus, Candida albicans, and Bacillus athrophaeus (formerly B. subtilis).

Stability also can be determined based on the reduction in the amount of endotoxins (e.g. lipopolysaccharides), growth factors, cytokines and other proteins and lipids present in a sample following exposure to the disinfectant solution.

### EXAMPLES

### Example 1

### Screening Stability of Different Colorants with ORP by UV-Vis

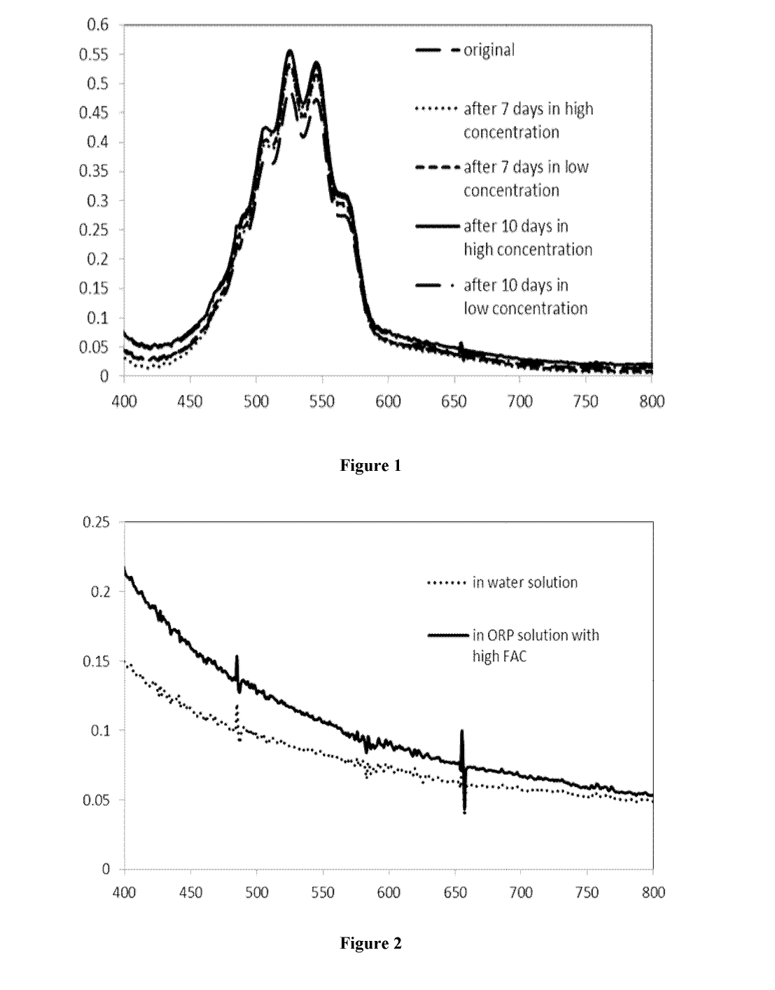

At 25° C. KMnO₄ (final concentration of KMnO₄ is 300 ppm) was added in two different ORP solutions: the high concentration one containing 100 ppm FAC (from Innovacyn, Inc.) and the low concentration one containing 30 ppm FAC. The UV-vis spectra of disinfectant solution were record by ultraviolet-visible spectrophotometer (HP 8453 HP 8453 PDA (G1103A) Spectrophotometer, wave length: 400-800 nm). Then the solution was stored in 25° C., UV-vis spectra was recorded after 7 days and 10 days. The UV-visible absorbance spectra demonstrated that when KMnO₄ was added in the ORP solution, its absorption did not decrease after 7 days and 10 days in both high FAC and low FAC concentration solutions when compared with the original KMnO₄ water solution (see FIG. 1).

To determine the stability of other colorants, colorant was dissolved to the above high concentration ORP and/or the above low concentration ORP separately at 25° C. and the final concentration of colorant in the solution was 300 ppm. The formed solution was the “disinfectant solution”. The UV-vis spectra of the “control solution” (as control, colorant was dissolved to the water separately and the final concentration of colorant in the solution was 300 ppm) and the “disinfectant solution” was recorded by above ultraviolet-visible spectrophotometer immediately after dissolved. If the stability is acceptable, the UV-vis spectra were further recorded after 10 days.

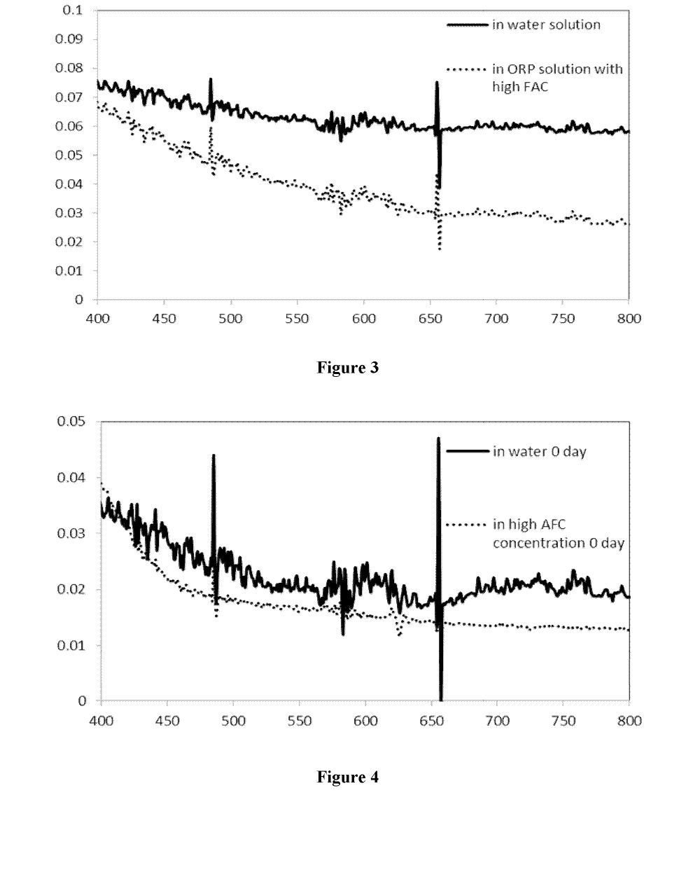

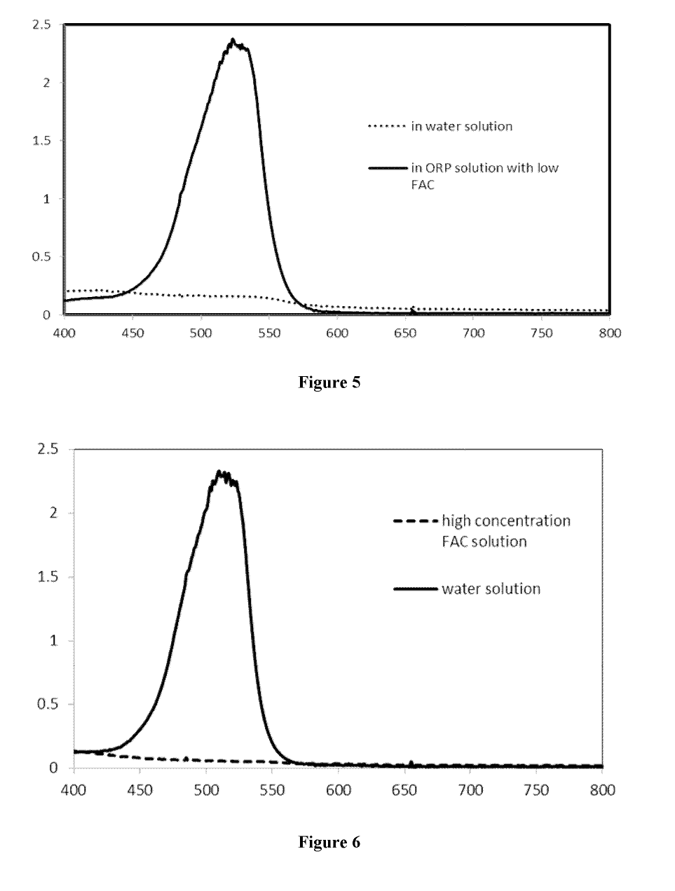

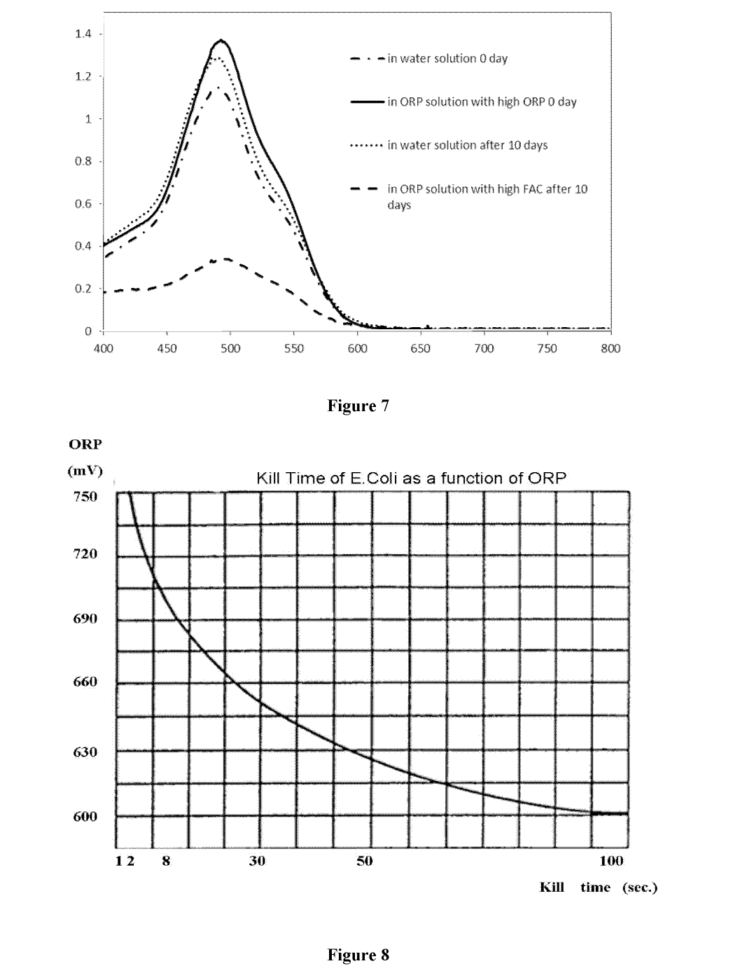

The stability of other compounds including iron chloride (see FIG. 2, light brownish color disappeared and white powder precipitated out.), Copper (II) Sulfate pentahydrate (see FIG. 3, light blue color disappeared and white powder precipitated out), ammonium ferric (iron III) citrate (see FIG. 4, light brownish color disappeared and white powder precipitated out), Eosine Free Acid Solvent Red 43 D&C red 21 (see FIG. 5, the red color disappeared quickly), Eosine YS Acid Red 87 D&C red 22 (see FIG. 6, the red color disappeared quickly) and Food dye FD&C red 40 (see FIG. 7, the UV-visible absorbance of the red 40 colored ORP solution decreased significantly after 10 days) were tested.

Potassium permanganate was very stable in the ORP water and the color intensity didn't decrease after storing for 10 days. All these compounds are water soluble and have relatively stable structures that are not easily oxidized by free active chlorines. However, the UV-vis spectra of their water solution and ORP water solution show that none of these compounds can survive in ORP water. Among these compounds, 300 ppm Copper (II) Sulfate pentahydrate, iron chloride, ammonium ferric (iron III) citrate water solution only gave very light blue or brownish color, thus they are not very good candidate as indicative colors for solutions. Furthermore, when these compounds were added inside ORP water solution, light blue to white powder precipitated out. The rest three red compounds gave very vibrant red color in water solution; however the color was gradually bleached in ORP water upon time.

Conclusion: Among all the tested colorants (natural dyes or synthesized dyes) and colored compounds, KMnO₄ is the only one can survive in the ORP solution. It can serve as the colorant for the ORP solution.

### Example 2

### Assay for Stability of Disinfectant Solution

ORP solution containing 6% sodium hypochlorite was prepared by reacting dilute sodium hydroxide solution with liquid or gaseous chlorine, accompanied by cooling. The basic principle of operation is to put water into a tank and add 50% sodium hydroxide until the strength of the caustic is approximately 6.75% (typical). Within a few batches, the amount of water and amount of sodium hydroxide to be added will be established and then lines may be drawn on the tank to show the operators how much water, and then sodium hydroxide, to add.

The 50% sodium hydroxide can be pumped into the tank or pulled into the system from the shipping container or a storage tank by partially closing the recycle tank outlet valve and opening the 50% caustic tank storage valve. After the sodium hydroxide is mixed, chlorine is added to the solution to react with the sodium hydroxide, except for a small amount of excess (0.2% by weight typical).

Control solution was obtained by directly diluting 2 mL fresh prepared ORP solution containing 6% sodium hypochlorite with deionized water to 1000 mL.

Sample 1 was obtained by adding 2 mL fresh prepared ORP solution containing 6% sodium hypochlorite, 50 ppm potassium permanganate, 15.5 g NaH₂PO₄ and 84.5 g Na₂HPO₄ into a 1 L volumetric flask and then diluting to 1000 mL Colored disinfection solution with deionized water. The pH value of the obtained solution is 7.5.

The ORP value for both control solution and sample 1 at different time period were measured by an ORP probe which is connected to an HQ440d benchtop multimeter (from Hach).

While the ORP value for the control sample gradually decreased, the ORP value for the disinfectant solution barely changed.

TABLE 1

| | | After 2 | | | |
|---|---|---|---|---|---|
| | | weeks | After 1 | After 2 | After 3 |
| | Initial | ORP | month | months | months |
| samples | ORP value | value | ORP value | ORP value | ORP value |
| Control 1 | 792.4 mV | 757.0 mV | 745.6 mV | 714.6 mV | 748.6 mV |
| Sample 1 | 810.5 mV | 811.1 mV | 810.3 mV | 814.4 mV | 818 mV |

Conclusion: KMnO₄ can also stabilize the original ORP solution, and the stability of the disinfectant solution is improved when comprising KMnO₄.

1. A disinfectant solution, comprising an oxidative and reductive potential (ORP) solution and a colorant.

2. The disinfectant solution of claim 1 wherein the colorant is dissolved in the ORP solution.

3. The disinfectant solution of claim 1, wherein the colorant renders the disinfectant solution a visible color.

4. The disinfectant solution of claim 1, wherein the colorant is potassium permanganate.

5. The disinfectant solution of claim 4, wherein the concentration of potassium permanganate is between a minimal value and a maximal value, wherein the minimal value is about 1 ppm and the maximal value is about 10%.

6. The disinfectant solution of claim 5, wherein the minimal value is selected from the group consisting of 1 ppm, 10 ppm, 20 ppm, 30 ppm, 40 ppm, 50 ppm, 60 ppm, 70 ppm, 80 ppm, 90 ppm, and 100 ppm.

7. The disinfectant solution of claim 5, wherein the maximal value is selected from the group consisting of 10%, 9%, 8%, 7%, 6%, 5%, 4%, 3%, 2%, 1%, and 2000 ppm.

8. The disinfectant solution of claim 5 wherein concentration of potassium permanganate is about 1-2000 ppm, about 10-2000 ppm, about 20-2000 ppm, about 30-2000 ppm, about 40-2000 ppm, about 50-2000 ppm, about 60-2000 ppm, about 70-2000 ppm, about 80-2000 ppm, about 90-2000 ppm, or about 100-2000 ppm.

9. The disinfectant solution of claim 1, wherein pH value of the disinfectant solution is between about 3 and about 9.

10. The disinfectant solution of claim 9, wherein pH value of the disinfectant solution is between about 6 and about 8.

11. The disinfectant solution of claim 10, wherein pH value of the disinfectant solution is between about 6.5 and about 7.5.

12. The disinfectant solution of claim 1, wherein the potential of disinfectant solution is between +500 mV and +1250 mV.

13. The disinfectant solution of claim 12, wherein the potential of disinfectant solution is between +600 mV and +1100 mV.

14. The disinfectant solution of claim 13, wherein the potential of disinfectant solution is between +700 mV and +800 mV.

15. The disinfectant solution of claim 1, wherein the ORP solution contains at least one species having disinfectant ability.

16. The disinfectant solution of claim 15, wherein the species is chlorine, bromine or ozone.

17. The disinfectant solution of claim 16, wherein chlorine is free available chlorine.

18. The disinfectant solution of claim 17, wherein the concentration of the free available chlorine is about 5ppm to about 6%.

19. The disinfectant solution of claim 17, wherein the concentration of the free available chlorine is about 20 ppm to about 1000 ppm.

20. The disinfectant solution of claim 1 further comprising an additive or a bleaching agent.

21. The disinfectant solution of claim 20, wherein the additive is selected from the group consisting of surfactant, detergent, cleaning agent, perfume, and scent-producing agent.

22. The disinfectant solution of claim 20, wherein the bleaching agent is chlorine-containing bleaching agent.

23. The disinfectant solution of claim 22, wherein the chlorine-containing bleaching agent is selected from the group consisting of chlorine, hypochlorites, N-chloro compounds, chlorine dioxide, sodium hypochlorite, hypochlorous acid, calcium hypochlorite, bleach liquor (aqueous solution of calcium hypochlorite and calcium chloride), bleaching powder (mixture of calcium hypochlorite, calcium hydroxide, calcium chloride, and hydrates thereof), dibasic magnesium hypochlorite, lithium hypochlorite, chlorinated trisodium phosphate and any combination thereof.

24. The disinfectant solution of claim 21, wherein the pH of the bleaching agent is from about 8 to about 10, and the potential of the solution is from about +700 mV to about +800 mV.

25. The disinfectant solution of claim 1, wherein the disinfectant solution is stable up to about 90 days, about 180 days, about 1 year, or about 2 years.

26. The disinfectant solution of claim 1, wherein the solution can be in the state of liquid, solid, aerosol, slurry or gel.

27. The disinfectant solution of claim 1, wherein the solution is packaged or sealed in a container.

28. The disinfectant solution of claim 1, wherein the OPR is in a first container and the colorant is in a second container.

29. Use of the disinfectant solution of claim 1, thereof in killing microorganisms, disinfecting surfaces and milking equipment; working as anti-infective agent; blocking inflammatory process, speeding the healing of burns, wounds, and diabetic ulcers; or treating periodontal diseases and skin disorders.

30. Use of the disinfectant solution of claim 1, thereof in medical device sterilization, food sterilization, hospitals, consumer households, agriculture, or anti-bioterrorism.

31. A method of forming the disinfectant solution of claim 1, comprising the steps of 1) providing oxidative and reductive potential (ORP) solution, and 2) dissolving a colorant into ORP solution.

32. A disinfectant article, comprising a substrate and the disinfectant solution of claim 1.

33. The disinfectant article of claim 32, wherein the substrate can be nonwoven materials, woven materials, compound materials, or knit materials.

34. The disinfectant article of claim 32, wherein the solution is dispensed, impregnated, coated, covered or applied to the substrate.

35. The disinfectant article of claim 32, wherein the solution is packed in a first container and the substrate is placed in a second container.

36. The disinfectant article of claim 32 further comprising a dispenser wherein the article is packed in the dispenser.
